(19) United States
(12) Patent Application Publication
(10) Pub. No.: US 2004/0202728 A1
(43) Pub. Date: Oct. 14, 2004

(54) Method for the destruction of oocysts

(76) Inventors: Sivaraj Shanker, New South Wales (AU); Heriberto Alejandro Bustamante, Miranda (AU); Marilyn Karaman, Aranda (AU); Richard Mark Pashley, Australian Capital Territory (AU)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/477,839

(22) PCT Filed: May 17, 2002
(86) PCT No.: PCT/AU02/00609

(30) Foreign Application Priority Data
May 18, 2001 (AU) PR 5128

(51) Int. Cl. A61K 33/08

(52) U.S. Cl. 424/690

(57) ABSTRACT

A method is provided for destroying oocysts, and destroying or inactivating sporozoites contained within the oocysts, by contacting the oocysts with a composition comprising surface-exposed AlOH groups to cause (a) the oocysts to rupture and the contained sporozoites to be released and/or (b) the oocysts to flatten resulting in destruction of the contained sporozoites. Also provided are pharmaceutical compositions, food supplements and items of clothing comprising a composition comprising surface-exposed AlOH groups.

### FIELD OF THE INVENTION

The present invention relates to a method for the destruction of oocysts and the killing of sporozoites.

### BACKGROUND TO THE INVENTION

Cryptosporidium parvum is a highly infectious protozoan parasite found in a wide range of animal hosts. It completes its life cycle within the gastrointestinal tract of a single host, the transmission from the infected host occurring via the excretion of faeces containing environmentally robust spheroidal oocysts of 4-6 μm in diameter. The Cryptosporidium oocyst wall is both chemically and physically tough and is virtually impervious to chemical disinfection with common disinfectants such as chlorine. The infected host can excrete large numbers (e.g. 10¹⁰ oocysts daily for up to 14 days) into the environment. The ubiquitous nature of Cryptosporidium favours catchment contamination and waterborne transmission.

In many cases the Cryptosporidium contaminated faeces reach water bodies such as dams and rivers. Ingestion of C. parvum-contaminated water may cause abdominal cramps, nausea and acute, self limiting diarrhoea that could last for 10-15 days in those with a normal immune system. However, Cryptosporidium ingestion by both the immunocompromised (e.g. the elderly, young infants, HIV/AIDS patients), or those receiving immunosuppressive therapy can potentially have complications with fatal consequences.

There is currently no therapeutic agent for cryptosporidiosis. The only option is symptomatic treatment. Cryptosporidium parvum has been responsible for between 250-500 million infections annually in Asia, Africa and Latin America. This ubiquitous protozoan is now a major concern to water and health authorities in developed countries due to its association with waterborne outbreaks of diarrhoeal disease.

Thus, the presence of Cryptosporidium in water is one of the more serious problems faced by the water treatment industry. The removal of Cryptosporidium parvum from potable water supplies has come under increasing legislative scrutiny in developed countries in the last few years.

During treatment of potable water, Cryptosporidium oocysts may be removed by conventional water treatment processes. These processes involve coagulation with coagulants such as ferric chloride or alum followed by addition of polyelectrolytes as coagulant aids and in some cases high molecular weight polymeric organic filter aids. The coagulated material is removed by either sedimentation or filtration through sand filters.

However, oocysts can break through, even when the plant is apparently operating under optimum conditions. The reasons for plant breakthrough are unclear. When this happens, oocysts reach the water reticulation system. This situation is aggravated because the oocysts, unlike other microorganisms, are resistant to chlorine disinfection normally carried out in water treatment plants and can remain viable for up to six months in water. Therefore, if oocyst breakthrough occurs in the water treatment plant it is likely that the water consuming population could be at risk.

C. parvum has also been known to contaminate public swimming pools. Several outbreaks of Cryptosporidiosis due to contaminated swimming pools have been reported. The contamination is usually due to faecal accidents in the pool and the spread of infection amongst pool users can be rapid. This is partly due to the ineffectiveness of current disinfection procedures.

Normally in swimming pools and spas, the water is continuously filtered through sand filters to remove particulate material and disinfected by chlorine addition followed by recirculation. In some instances low concentrations of inorganic coagulants are added to optimise solids removal. However, if Cryptosporidium contamination occurs, removal by filtration or coagulation/filtration through sand filters may not be completely effective. Detection of oocysts results in pool closure for the treatment of the water, for example, by superchlorination at a level of 3-5 mg/L. The efficiency of superchlorination as treatment for deactivating Cryptosporidium is not guaranteed. The use of other stronger disinfectants such as ozone, chlorine oxide or mixtures of strong oxidants has also been tested with mixed success.

Accordingly, a need exists for a more effective and reliable means for removing Cryptosporidium contamination from various forms of water supplies such as potable water supplies and recreational water supplies.

### SUMMARY OF THE INVENTION

We have found that compositions comprising surface-exposed AlOH groups are capable of not only capturing oocysts irreversibly but also destroying the oocyst structure due to the strength of the specific adhesive force between the oocyst's surface and the AlOH groups. This results in the release of the sporozoites contained within the oocysts which are then more susceptible to killing by anti-sporozoidal agents. Furthermore, the sporozoites may themselves be adsorbed to the surface-exposed AlOH groups. It is also possible that the sporozoites are also destroyed inside the oocysts when the oocysts are flattened as a consequence of the adsorption process.

Accordingly the present invention provides a method of destroying oocysts, and destroying or inactivating sporozoites contained within the oocysts, which method comprises:

(i) contacting the oocysts with a composition comprising surface-exposed AlOH groups to cause (a) the oocysts to rupture and the contained sporozoites to be released and/or (b) the oocysts to flatten resulting in destruction of the contained sporozoites; and, optionally

(ii) contacting the released sporozoites with an effective amount of an anti-sporozoidal agent.

Preferably in (a), the oocysts adsorb on the surface-exposed AlOH groups, resulting in the adsorbed oocysts rupturing and the contained sporozoites being released.

In another embodiment, the present invention provides a method of destroying oocysts, and destroying or inactivating sporozoites contained within the oocysts, which method comprises:

(i) contacting the oocysts with a composition comprising surface-exposed AlOH groups which adsorb the oocysts causing the adsorbed oocysts (a) to rupture and the contained sporozoites to be released and/or (b) to flatten resulting in destruction of the contained sporozoites; and, optionally

(ii) contacting the released sporozoites with an effective amount of an anti-sporozoidal agent

The present invention also provides a method of destroying an oocyst, and destroying or inactivating one or more sporozoites contained within the oocyst, which method comprises contacting the oocyst with a composition comprising surface-exposed AlOH groups to cause the oocyst to rupture and the contained sporozoites to be released such that the released sporozoites are brought into contact with the surface-exposed AlOH groups. The released sporozoites may optionally be contacted with an effective amount of an anti-sporozoidal agent.

Preferably the surface-exposed AlOH groups are present as hydrated alumina.

Typically, one or more of the oocysts are Cryptosporidium oocysts and/or Giardia cysts, more preferably Cryptosporidium oocysts.

The oocysts may be present in a fluid, such as a biological fluid or water. In one embodiment, the fluid may be purified to remove the anti-sporozoidal agent and/or composition

The water may be intended for use by humans or animals, such as for consumption by humans or animals. The water may also be intended for the preparation of food products or pharmaceutical products.

The present invention further provides a pharmaceutical composition comprising a composition comprising surface-exposed AlOH groups together with a pharmaceutically acceptable carrier or diluent. Also provided is a method for producing the pharmaceutical composition which method comprises admixing a composition comprising surface-exposed AlOH groups with a pharmaceutically acceptable carrier or diluent.

The present invention also provides a food supplement comprising a composition comprising surface-exposed AlOH groups.

In a further aspect, the present invention provides a method of treating, preventing or reducing a protozoal infection in a human or animal, said protozoal infection being initiated by the ingestion of oocysts, which method comprises administering to said animal or human a pharmaceutical composition of the invention or a food supplement of the invention. The method may further comprise administering an anti-sporozoidal agent to the animal or human.

In another aspect, the present invention provides a food receptacle, drinking receptacle or drinking straw comprising a composition comprising surface-exposed AlOH groups such that when in use, the composition contacts food or fluid placed in the receptacle or drawn through the drinking straw.

The present invention also provides an item of clothing selected from a nappy, underwear or swimwear comprising a composition comprising surface-exposed AlOH groups such that when the clothing is worn by an individual, the composition is brought into contact with faecal matter produced by the individual. The item of clothing may further comprise an anti-sporozoidal agent.

The present invention further provides a method of destroying or inactivating a pathogenic proteinaceous agent which method comprises contacting the agent with a composition comprising surface-exposed AlOH groups to cause the agent to be adsorbed to the surface-exposed AlOH groups.

### DETAILED DESCRIPTION OF THE INVENTION

### Compositions or Mediums Comprising Surface-exposed AlOH Groups

Compositions for use in the present invention comprise AlOH groups which are exposed at the surface of the composition. A preferred composition is one which comprises alumina (Al₂O₃) which is hydrated at the surface so as to form surface Al—OH groups. This material presents a chemically active substrate for the direct adsorption and destruction of suitable biological species such as protozoal oocysts. It is, however, important that the alumina is in the appropriately hydrated form. The compositions may comprise other materials that do not themselves comprise AlOH groups provided that the surface of the composition has a suitable density of exposed AlOH groups.

The surface density of Al—OH groups on the surface of the composition is ideally an average of greater than about 1 hydroxyl group per 10 nm² of surface (1 hydroxyl group per 10 nm²), preferably greater than about 1 hydroxyl group per 5 nm², or 1 hydroxyl group per 3 nm² especially 1 hydroxyl group per 2 nm². Most preferably, the density of the surface hydroxyl groups is an average of greater than about 1 hydroxyl group per 1 nm, especially greater than about 1 hydroxyl group per 0.75 nm² or about 1 hydroxyl group per 0.5 nm². When the Al₂O₃ surface is essentially fully hydrated, thereby providing a maximized surface area available for adsorption of the biological species to be separated, the average rate of surface Al—OH groups per nm² of surface area, is about 1 hydroxyl group per 0.18 nm² to about 1 hydroxyl group per 0.25 nm². In general terms, fully hydrated alumina is most effective for the removal and destruction of oocysts.

The introduction of surface Al—OH groups onto activated alumina is thermodynamically favoured and can be achieved by hydrating methods known to those skilled in the art, for example activated alumina may be soaked with water for a prolonged time. A second method involves treatment with sodium hydroxide (NaOH), where the upper alumina surface is dissolved thus allowing other hydroxyl groups to be formed. In a third method, the activated alumina may be treated by exposure to ultraviolet light in the presence of water vapour. This process produces ozone which breaks the Al—O—Al bond allowing the formation of Al—OH. In a fourth method activated alumina is treated with hydrogen peroxide which produces a hydroxyl radical which attacks the Al—O—Al bond allowing the formation of Al—OH. These methods may be controlled to introduce the desired frequency of Al—OH groups over the surface area. By way of example only, the alumina surface may be hydroxylated by treatment of the alumina in 1×10⁻²M NaOH or in 30% w/v/H₂O₂ for more than one hour or treatment with ozone in the presence of water vapour.

Because of the nature of the alumina surface, activated alumina (dehydrated alumina) still contains some hydroxylated sites, for example less than about 1 hydroxyl group per 10 nm². However, this material is ineffective in removal of Cryptosporidium from contaminated water.

The term “composition” includes a range of physical forms including powders, granules, crystalline solids, plates, or compressed discs or wafers.

The alumina may exist in the amorphous state or as alpha-Al₂O₃ or gamma-Al₂O₃.

Particulate alumina, such as powdered and granulated forms, provide an increased surface area per volume and are suitable for packaging into cartridges which can be used alone or in conjunction with other filtration systems. Powdered and granular alumina is readily available in different diameter size ranges for example, from about 15 mm down to about 50 microns (0.05 mm). The size of the particulate alumina used may be varied depending on the application. By way of example only, one particulate size range contemplated by the invention is from about 5 mm to about 1 mm for example, about 3-2 mm. Another particulate size range is from about 1.5 mm to about 0.5 mm. Yet another particulate size range contemplated by the present invention is from about 0.5 mm to about 0.05 mm, for example 0.3 mm to about 0.1 mm.

Depending on the application generally the particle sizes will be between 500 microns (0.5 mm) to 13 mm. The most suitable size range will be selected in terms of effective size and uniformity coefficient.

In the case of municipal water treatment, usually larger particles size, typically greater than 1 mm would be preferred in order to achieve appropriate water throughput. However, pilot plant testing may be carried out to establish the optimum relationship between the thickness of the alumina bed and the particle size to ensure maximum removal whilst maintaining high water throughput.

Similarly in the case of water treatment for industrial purposes, such as in the preparation of water for use in the manufacture of food and pharmaceuticals, relatively large volumes of water will be treated. Accordingly, a similar approach to municipal water will usually be adopted. It must, however, be realised that the use of filter cartridges containing the hydrated alumina may be desirable in some manufacturing facilities. In both municipal and private swimming pools applications it may be appropriate to use finer particles, say between 0.5-2 mm to maximise collision and capture of biological species by the particles.

In the purification of domestic water it would be appropriate to use smallest particle sizes to both minimise the size of the filter device and to achieve maximum surface area whilst ensuring that pressure drops across the filter cartridge containing the alumina are minimised.

### Methods of Destroying Oocysts and Sporozoites

The above compositions can be used in methods of destroying oocysts and the sporozoites contained within the oocysts. Previous methods used to kill microorganisms have been ineffective against protozoal oocysts because the oocyst wall is both chemical and physically tough. Thus the sporozoites contained within the oocysts are protected from chemical disinfectants. However, the present invention provides a method for killing the sporozoites by adsorption of oocysts to surface-exposed AlOH groups which results in rupture of the oocyst wall. The released sporozoites may then be contacted with anti-sporozoidal agents such as chlorine or ozone. Other anti-sporozoidal agents include strong disinfectants, gamma irradiation, UV, chlorine derivatives including chloramine, chlorine oxide, mixed oxidants, or mixtures of strong oxidants, ultrasound, high temperature. Furthermore, the surface Al—OH groups are also capable of adsorbing and destroying the released sporozoites. In addition, the adsorption of the oocyst to the surface Al—OH groups may result in flattening of the oocyst and destruction of sporozoites contained within the oocyst.

The terms “adsorb” and “adsorption” as used herein may refer to either electrostatic adsorption or chemisorption.

Chemisorption is an irreversible form of adsorption resulting from the formation of chemical bonds between surface sites on approaching surfaces. In natural systems chemisorption typically occurs between carboxylate, phosphate and wide range of metal cations such as aluminium, calcium, iron etc. The precise nature of these chemical interactions is often complex but may involve ligand bonding to carboxylate and phosphate groups on the surface of micro-organisms.

In addition, van der Waals forces generally act to pull colloids together into strong adhesive contact. Oppositely charged colloids usually adhere due to electrostatic forces.

The oocysts are typically present in a fluid which it is desired to treat to remove oocyst contamination. Particular types of fluids include water and biological fluids such as blood, plasma, urine and bodily secretions. The oocysts may also be present in solids, such as solid food or faeces, particularly solids with a substantial water content.

Water may be obtained from a variety of sources and may be used for a variety of purposes. For example, the water may be untreated or partially treated sewage, swimming pool water or treated drinking water. The water may subsequently be used for consumption by humans or animals, the preparation of food products or pharmaceutical products. The water may also be used in aquaculture e.g. filling of fish ponds or tanks, depuration of oysters, so that the aquatic organisms do not become contaminated with or retain pathogenic organisms.

The method of killing oocysts according to the present invention comprises contacting the oocysts with the composition of the invention. Typically, the oocysts are brought into contact with the composition by the flow of a fluid containing the oocysts towards, over or through the composition, such as in the case of a water treatment plant, swimming pool treatment apparatus or other means comprising a means for producing a flow of fluid. Alternatively, the composition may be added to a fluid containing the oocysts, for example by the addition of a powder or granules to a fluid or by administration of the composition as a pharmaceutical composition to an individual infected with oocysts or sporozoites.

Adsorption of the oocysts on the composition then results in rupture of the oocyst wall, destroying the oocyst and releasing the sporozoites contained therein. The sporozoites may then be brought into contact with the surface-exposed AlOH group which may also result in their destruction and/or physical removal from the fluid or solid which is being treated. Alternatively, or in addition, the released sporozoites may be contacted with an effective amount of an anti-sporozoidal agent which inactivates or kills the sporozoites. The anti-sporozoidal agent may for example, be present as part of the composition of the invention or present in the fluid to be treated. When present in the fluid to be treated, the anti-sporozoidal agent may be present before, during and/or after the step of contacting the oocysts with a composition of the invention. Thus, for example, treated water after being contacted with a composition of the invention may no longer comprise viable oocysts but may still contain some released sporozoites. Treatment at that stage with an anti-sporozoidal agent can be used to kill such sporozoites.

Furthermore, although some fluids prior to contact with the compositions of the invention may have already been treated with agents that also function as anti-sporozoidal agents, the remaining levels of the agent may be too low and may need to be boosted to ensure levels effective in killing the released sporozoites.

In some applications, it may be desirable to further purify treated fluids etc. to remove the anti-sporozoidal agent and/or the composition, such as when the fluids are to be used for pharmaceutical or medical purposes. Suitable treatments for the removal of disinfectants such as chlorine are known in the art (e.g. activated charcoal resins).

For the majority of applications, the contact time between the composition comprising surface-exposed AlOH groups and the fluid or solid to be treated will be minimal. Typically contact times of between about 1, 2 or 5 seconds and 1 hour will be sufficient to achieve normal removal. The contact time is, however, dependent on a variety of factors applicable to each use situation such as the extent of the contamination, the available surface area of composition for contact with the water, i.e. particle size and volume of composition, the surface density of hydroxyl groups and the flow rate of water over or through the composition. The person skilled in the art will appreciate that a suitable contact time may be established through appropriate testing and evaluation. Techniques for monitoring oocyst destruction using microscopy are outlined in the Examples.

In general terms, operation of the invention will typically result in at least a 1 log reduction in the oocysts present in the water. In the context of this specification, a log reduction refers to a 10-fold reduction. For example, if there were 1000 oocysts per ml in a water sample, a 1 log reduction would result in 100 oocysts remaining. A 2 log reduction would result in 10 oocysts remaining. Preferably there will be a 2 log reduction, desirably a 3 log reduction, most preferably a 4 log reduction. It is especially preferred that the invention operates to such that there is at least a 5 log reduction, particularly a 6 log reduction.

Removal of the proportion of the biological species may be achieved in one treatment or, optionally the process of contacting the contaminated fluids solids with the Al—OH surface may be repeated to provide the desired level of removal of the oocysts or other biological species from the fluids or solids.

### Uses

The methods of the invention may be used in any application where it is desired to kill/destroy oocysts. Some possible uses are shown in FIG. 1. The term “oocyst” refers to the cyst formed around two conjugating gametes in certain protozoa and in Sporozoea (a Class of protozoa), the passive phase into which the active vermiform phase changes in the host. Preferred species which form oocysts or cysts which may be killed or destroyed by the methods of the present invention are Cyptosporidium and Giardia, preferably Cyptosporidium and any other protozoa (e.g. Cyclospora, Caryospora, Isospora, microsporidia and Blastocystis).

The methods of the present invention may be used to treat drinking water/sewage effluent In this embodiment, the composition comprising surface-exposed AlOH groups may be part of a mixed filter bed. In this form, the composition is generally disposed on the downstream side of the in flowing water. In this way, the water will preferably have been conventionally treated prior to contacting the composition. The person skilled in the art will appreciate that the mixed filter bed may include discrete beds of, for example, hydrated alumina of different particle size ranges.

It is also important to appreciate that in some applications, it may be permissible to utilise beds of the composition comprising surface-exposed AlOH groups that are fed under gravity.

In order to maximise the adsorptive capacity of the composition for biological species, preferably the bed will be used as polishing filter. Thus, in some embodiments of the present invention it is envisaged that the compositon will be used as a separate polishing “monofilter” after the conventional filters that remove the flocs from the flocculated raw water.

In this configuration it is easier to take the filter off-line when it is exhausted in order to chemically regenerate the composition. It must be recognised that there may be some applications where the composition may be used with little or no pretreatment of the in flowing water.

Prior to contacting the water with the composition, in the case of the treatment of municipal water, both turbidity and colour are usually removed by the addition of suitable inorganic coagulants and organic polyelectrolytes. If the municipal water is hard, preferably the water will be softened by lime softening, lime-soda ash softening or excess-lime treatment.

Furthermore, the composition may be used for the treatment of the supernatant of the backwash water, for example in the preparation of municipal water, thus ensuring that organisms such as Cryptosporidium are removed. Backwash water is generated in water treatment plants by reversing the water flow through a filter in order to remove the material trapped. The backwash water is normally decanted and the supernatant may be recycled to the head of works.

In a domestic water situation, the water will have already have been treated by the normal processes as described above. However, viable micro-organisms may remain in the water supply or may be introduced between the water treatment plant and the domestic user. This may be due to sewage infiltration into the reticulation system.

Whilst swimming pool water is not classified as potable water it is important that microbial contamination and the consequent risk to public health is minimised. This is particularly important in the case of public swimming pools and spas. In order to maintain water quality, it is desirable that swimming pool water is subjected to filtration and disinfection. As chlorine is inefficient as a disinfectant against Cryptosporidium it is important to be able to remove it from the swim pool water as the water is being filtered prior to recirculation.

An advantage of the present invention is that it may be readily utilised as an adjunct to existing water treatment facilities. As mentioned above, in most applications, the hydrated alumina bed will be used as a final polishing filter. This permits an existing water treatment facility to be upgraded by retro-fitting an additional stage after the current water treatment stages.

The comprising surface-exposed AlOH groups, preferably hydrated alumina, may be packed into a suitable, high flow rate filtration cartridge and may, for example, be used as the final stage in a swimming pool pumping-filtration unit. Alternatively, such cartridges may be used directly in conjunction with a domestic water reticulation system. In this form, the cartridge may be fitted to tap(s) from which drinking water is to be obtained or to the inflow from the municipal water supply.

In a domestic situation, it may also be appropriate to use a bed of the composition within a gravity fed cartridge. In this situation, water is simply fed under gravity through a cartridge that is open to receive the water at one end and at the other end, allows the water to drain into a receiving vessel. Alternatively, the hydrated alumina may be contained in a water permeable bag. In this situation, the bag containing the hydrated alumina is immersed in a vessel of water to be treated for a suitable contact period.

Fluids, such as water, treated by the methods of the invention may be used in a variety of applications including the preparation of foodstuffs (including washing foodstuffs) and the preparation of pharmaceutical products.

Furthermore, the findings disclosed herein showing that compositions comprising surface-exposed AlOH groups may be used to kill/destroy oocysts and the sporozoites contained therein may be applied to the productions of prophylactic and therapeutic products for the prevention of and/or treatment of protozoal infections. Thus, a composition comprising surface-exposed AlOH groups may be used to prepare a pharmaceutical product by admixing with a pharmaceutically acceptable carrier or diluent Suitable carriers and diluents include isotonic saline solutions, for example phosphate-buffered saline. Alumina should be used as a fine (roughly micrometer size) particulate dispersion in aqueous solution. Unlike for filtration uses, the size range for aqueous delivery systems should be much finer so that the alumina remains dispersed in the aqueous phase long enough to be administered orally. The pharmaceutical composition of the invention may typically be administered by oral administration since oocyst-mediated infection is present primarily as a gastrointestinal tract colonisation (in the upper intestine).

The compositions comprising surface-exposed AlOH groups may also be administered as a foodstuff.

Typically, the composition may be administered at a dose of from 1 mg/kg body weight to 100 mg/kg body weight, preferably from 10 or 20 mg/kg to 100 mg/kg body weight.

The routes of administration and dosages described are intended only as a guide since a skilled practitioner will be able to determine readily the optimum route of administration and dosage for any particular patient and condition.

Thus, the present invention provides a method of treating, preventing or reducing a protozoal infection in a human or animal, said protozoal infection being initiated by the ingestion of oocysts, which method comprises administering to said animal or human a pharmaceutical composition of the invention or a food supplement of the invention. In some instances it may be desirable to co-administer an anti-sporozoidal agent to the animal or human to ensure that the released sporozoites are destroyed. Suitable anti-sporozoidal agents may include nitroimidazoles, co-trimoxazole, anti-malarial regimes, clindamycin, tetracycline, pyrimethamine/sulphadiazine.

Compositions of the invention may also be used in the manufacture of utensils and containers used for the consumption, presentation or storage of food such as a food receptacle, drinking receptacle or drinking straw. Such items will be manufactured such that a composition comprising surface-exposed AlOH groups is on a surface that is exposed to the liquid or food that is being consumed i.e. such that when in use, the composition contacts food or fluid placed in the receptacle or drawn through the drinking straw.

Furthermore, as discussed above, faecal accidents are a major cause of oocysts contamination of swimming pool water. One possible means to reduce contamination, especially in individuals known to present a risk of faecal accidents, such as babies and infants is to provide suitable clothing comprising a composition comprising surface-exposed AlOH groups such that when the clothing is worn by an individual, the composition is brought into contact with faecal matter produced by the individual. Items of clothing may include swimwear. In a more general context, it may be desirable to neutralise oocyst infections in faecal matter produced by infected individuals by providing underclothing that comprises a composition comprising surface-exposed AlOH groups such that when the clothing is worn by an individual, the composition is brought into contact with faecal matter produced by the individual. Underclothing includes underwear and nappies.

The above items of clothing may desirably further comprise an anti-sporozoidal agent The composition and optionally the anti-sporozoidal agent may typically be present in the item of clothing as a liner incorporating the composition.

In addition to oocysts, it may be possible to use compositions comprising surface-exposed AlOH groups to remove proteins from fluids or solids. Of particular interest are pathogenic proteinaceous agents such as prions. Fluids or solids may include biological materials such as blood, plasma, compositions comprising growth factors, animal derived products etc.

### Modes for Carrying out the Invention

The invention will now be described with reference to the following non-limiting Examples and Figures. In these examples, destruction of Cryptosporidium oocysts is described. It will of course be appreciated by persons skilled in the art that other organisms, particularly microbial pathogens may be removed from water using this invention.

### BRIEF DESCRIPTION OF DRAWINGS

FIG. 1. Showing possible uses of the technology described herein.

FIG. 2. Showing the zeta potential (ζ) of Cryptosporidium parvum oocysts as a function of pH. Note: Live (triangles), gamma-irradiated (squares) and formalinised (diamonds).

FIG. 3. Showing a contact mode AFM image of oocyst debris produced after adsorption of Cryptosporidium oocysts to a plasma cleaned sapphire (α-alumina) wafer.

FIG. 4. AFM image of the adsorbed oocyst debris to a plasma cleaned sapphire wafer.

FIG. 5. Showing confocal micrographs of silica and alumina substrates after exposure to Cryptosporidium oocysts.

FIG. 6. Graphically depicts the permeate levels of Cryptosporidium oocysts adsorbed after repeated washings through various packed columns of natural minerals: alumina (Al₂O₃), fluorspar (CaF₂), geothite (alpha-FeO.OH), rutile (TiO₂), pyrite (FeS₂) and silica (SiO₂).

FIG. 7. Schematic showing (A) a number of fine alumina particles in distilled water being released from packed alumina column (i.e. blank) and (B) showing a reduction in particulate released (<background levels) after oocyst loading. This suggest that the oocysts, actually reduce the release of particulate material from the column.

### EXAMPLES

### Example 1

Microelectrophoresis of Formalinised, Gamma-irradiated and Live Cryptosporidium Oocysts.

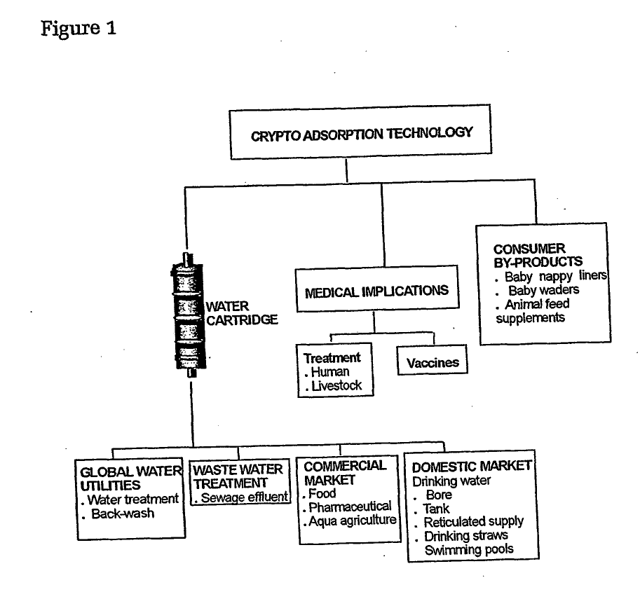

Previously, the zeta potential of gamma-irradiated Sydney isolate of Cryptosporidium parvum oocysts was found to be negative at pH values higher than around 2.7. The negative zeta potential increased its value up to around pH 6.0 and leveled off at pH values greater than 6.0 (refer FIG. 1). This data was fitted with a mass action model. From this the k_{aq} was estimated to be 2×10¹⁸ ions/cm³ (˜3.3×10⁻³) giving a pKₐ, of 2.5. This pKₐ value suggests that the ionizable groups contributing to the oocyst negative surface potential could be either carboxylate, phosphate or a combination of the two. The estimated pKₐ is between that reported for a terminal carboxylate in a protein of 3.1 and for an ionizable phosphate in a phospholipid of 2.1 or a carboxylic acid associated with a glycoprotein (e.g. sialic acid pKₐ 2.5).

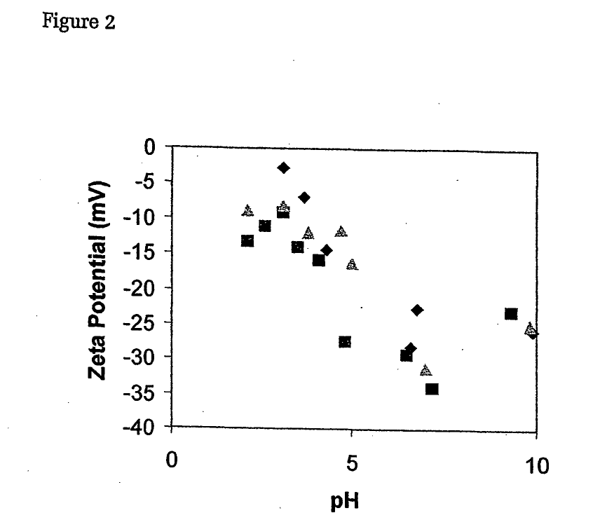

To determine the impact of chemical and physical inactivation on the charging behaviour of Cryptosporidium parvum oocysts, the charging behaviour of a pristine live sample processed and stored in the absence of any antibiotics or surfactants was determined. In addition, as column, adsorption and confocal investigations used a number of Cryptosporidium samples which were processed in a variety of ways, it was important to ascertain whether the results obtained by these investigations were in any way influenced by the inactivation (i.e. formalinised or gamma irradiated) process used. The live pristine sample was divided into several sub-samples and subsequently formalinsed, gamma-irradiated or treated with potassium dichromate, and the impact of such treatment was assessed using micro-electrophoresis. The results obtained suggested the surface charging properties for formalinised, gamma-irradiated samples were similar to the live sample (refer FIG. 2).

### Example 2

Rupture of Live and Formalinised Oocysts and Release of Sporozoites by Adsorption to Alumina (Optical and Atomic Force Microscopy Studies)

Oocyst samples obtained from Murdoch University (W.A) with a oocyst concentration of 6×10⁵ oocysts/ml were made inactive by treatment with (10%) formalin. The formalinised oocyst adsorption was carried out on two different substrates poly-D-lysine coated silicon and perfectly clean alumina wafers.

The poly-D-lysine coated silicon was used as a blank to demonstrate the specific effect of the alumina surfaces. A silicon wafer was coated with a thick layer of (0.1% w/v) poly-D-lysine (70,000-150,000 molar mass) and imaged under water using contact mode AFM.

The substrates immersed in aqueous media were contacted with water containing oocysts and the effect of the adsorption followed by both optical and atomic force microscopy.

The formalinised oocyst deposited onto the poly-D-lysine coated silica substrate was followed in real time using an optical microscope. The oocysts were seen to slowly settle under gravity onto the substrate and upon contact, the oocysts remained spherical and mobile indicating no adsorption.

Using contact mode AFM the oocysts were found to be only weakly adhered on the substrate. It was evident that the oocysts were unaffected by the weak adhesion process (i.e. remaining perfectly spherical, with no deformation of the oocyst shell evident).

Upon rastering the AFM stylus across the substrate, the oocysts were found to be easily moved around on the substrate surface using the AFM cantilever, even after more than 48 hrs, indicating that the oocysts were simply “resting” on the substrate. Note: No AFM image of the oocysts on poly-D-lysine could be obtained because of this mobility, since only fixed objects can be imaged. However, the mobile oocysts could be seen through an optical microscope and their behaviour was captured on videotape and photographed from the monitor.

The adsorption of the same formalinised oocyst sample onto the uncoated plasma oxidised alumina wafer substrate was followed in real time using an optical microscope. The oocysts upon adsorption became angular and irregular (no spherical objects were observed) and were much less well defined (even within only 10 minutes' exposure). In real time optical microscopy—captured on video tape—the oocysts were found to rupture immediately upon settling onto the substrate under gravity and the anchored debris was found to flicker rapidly, possibly as the sporozoites were released.

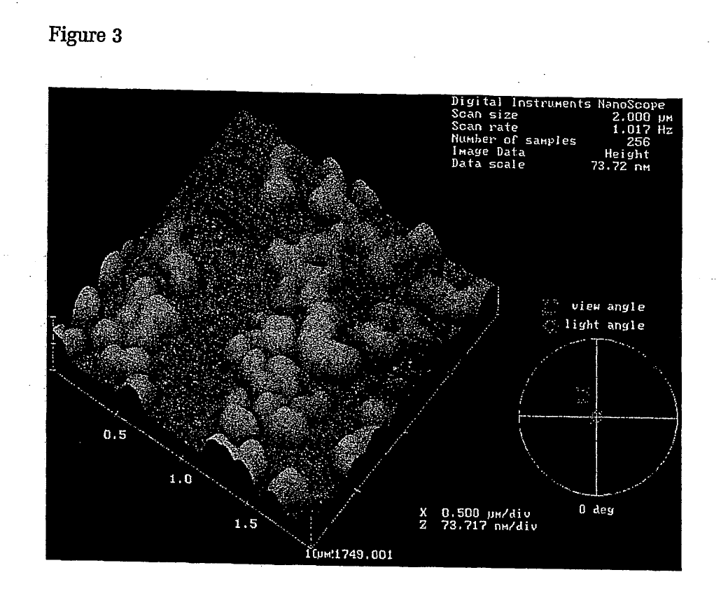

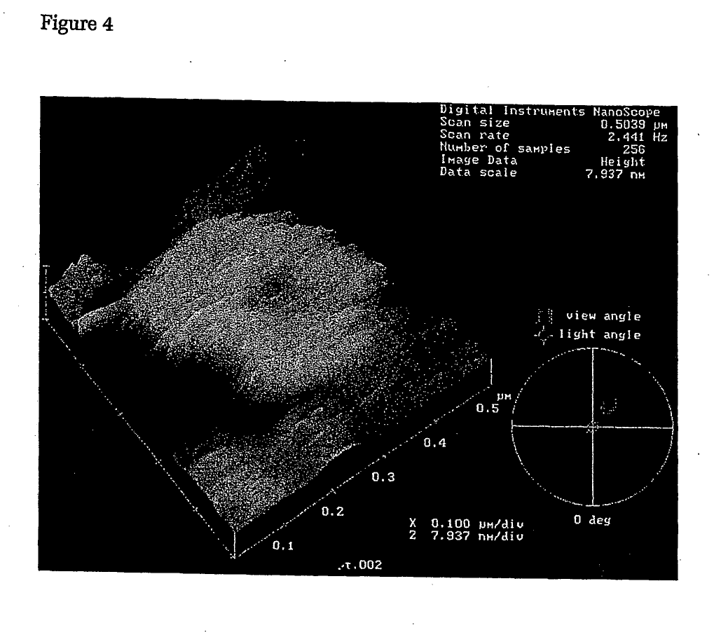

Closer examination using atomic force microscopy revealed oocyst debris, was strongly adsorbed to the substrate and clearly much smaller than the initial oocyst size (see FIGS. 3 and 4). On further examination using the AFM some of the debris appeared to be adsorbed sporozoites.

These experiments were repeated with gamma-irradiated and live Cryptosporidium oocysts—similar capture and destruction results were obtained.

Adsorption stability was tested by placing the sapphire (α-alumina) substrate with adsorbed oocysts into a solution at pH to 9.2 and 10 (i.e pH>iep). At this pH both the oocysts and the sapphire (α-alumina) wafer are negatively charged, therefore if the adsorption was purely electrostatic then desorption would result. This was found not to be the case; the substrate was soaked for several hours and no oocyst desorption was found to occur demonstrating irreversible chemisorption. There also appears every indication that the sporozoites are also strongly adsorbed onto the alumina surface.

These results suggest that upon adsorption the substrate not only captures the organism irreversibly but, destroys the oocyst structure due to the strong, specific adhesion between the active chemical groups on the oocyst surface and the Al—OH groups.

### Example 3

Adsorption to Silica and Alumina Substrates—a Comparison (Confocal Studies).

The adsorption of live and formalinised Cryptosporidium oocysts, after exposure to silica and alumina substrates were studied using a Biorad Radiance 2000 confocal microscope.

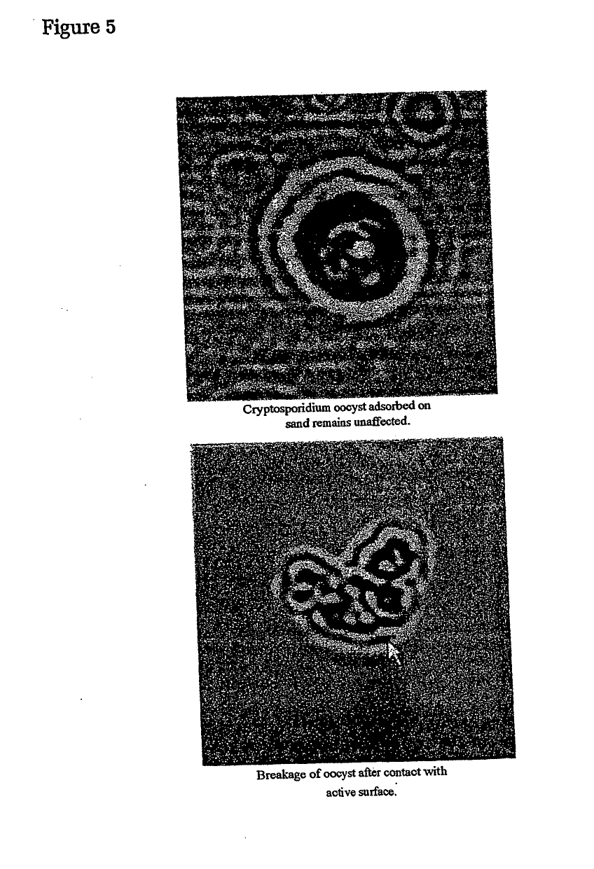

A 100 μl aliquot of a 6.8×10⁶ oocysts/ml sample of formalinized oocysts in solution (−28 mV @ pH 6.6) was transferred to either a welled, silica slide cover-slip sealed with nail varnish, or placed into a silica-quartz cell for confocal study. Oocysts adsorbed to the silica were found to be spherical (4.5 μm dia.) with characteristic internal structures reflecting the presence of sporozoites, were not deformed, were mobile and moved freely with no oocyst debris present (see FIG. 5).

By contrast, the same oocysts sample when exposed to plasma cleaned (0.01 torr Ar 0.05 torr H₂O, High 1 min) sapphire (α-alumina) wafers were found to have no defined oocyst shell upon adsorption. Oocyst shell debris was evident of approximately 2-2.5 μm in diameter, were non spherical and non mobile (see FIG. 5).

The confocal study was repeated using a live Sydney isolate (−30 mV @ pH5.6). Similar results were obtained to those for the formalinised oocysts. The destruction of the oocysts were found to be immediate and 100% effective.

To determine the efficiency and log removal, 100 mL aliquots of 1×10⁶ oocysts/ml were continually loaded in a cell containing a 2 mm diameter sapphire wafer. The destruction was found to be extremely fast and efficient, the capture/destruction was so fast that it could not be visualised in real time.

Repeated studies confirmed the destruction and efficiency of capture/destruction for both live and formalinized oocysts on sapphire wafers but not silica substrates.

### Example 4

Microelectrophoresis of Cryptosporidium Oocysts in the Presence of Water Treatment Coagulants—a Comparison of Ferric Flocculants and Alum Flocculants.

The zeta potentials of Cryptosporidium oocysts in the presence 5 mg/L ferric chloride were studied over a pH range 3-10. The measured potentials were found to be negative over the entire range and similar in magnitude and sign to oocysts in the absence of coagulant. This suggested no adsorption of soluble ionic hydrolysed iron species or primary ferric hydroxide precursor particles occurred over this pH range.

As with the ferric flocs, it was possible to differentiate between the alum flocs and the oocyst in solution because of their different size, shape and scattering ability. The zeta potentials of Cryptosporidium oocysts in the presence of 6.8 mg/L alum were studied over a pH range 4-8. The measured potentials were found to be similar in magnitude and sign to that of the alum flocs alone.

The specific adsorption of the hydrolysed aluminium based species in solution caused the oocyst potential to change sign below pH 7. At pH values above 7.5 the oocysts became increasingly more negatively. The magnitude and sign of the measured potentials of the entire pH range studied (pH 4-8) were similar to that of the alum flocs formed in the absence of oocysts. This suggested the hydrolysed aluminium based species in solution had chemisorbed to the oocyst surface facilitating the growth of Al(OH)₃ floc around the oocyst

These results provide support for the specificity of hydrolysed aluminium based species for protozoal, e.g. Cryptosporidium, oocysts.

### Example 5

Column Filtration Experiments

Alumina

The charging behaviour of Al₂O₃ powder used in the column experiments was determined as a function of pH by microelectrophoresis. The alumina was found to have a positive charge at pH values below 6. The Al₂O₃ surface charge approached zero (iso-electric point) around pH 6.1-6.2. At pH values above 6 the Al₂O₃ surface became negatively charged.

The surface reactions responsible for both the positive and negative charge have been described in the literature as follows:

MOH₂⁺^{_{—}}^{H+}→—MOH→^{OH—}—MO⁻+H₂O

Mⁿ⁺+xOH⁻→M(OH)ₓ^{(n-x)+}

M(OH)ₓ^{(n-x)+}+OH—→M(OH)ₓ₊₁^{(n-x-1)+}

M(OH)ₓ^{(n-x)+}+H⁺→M(OH)ₓ₋₁^{(n-x+1)}+H₂O—

Where M is the metal Al.

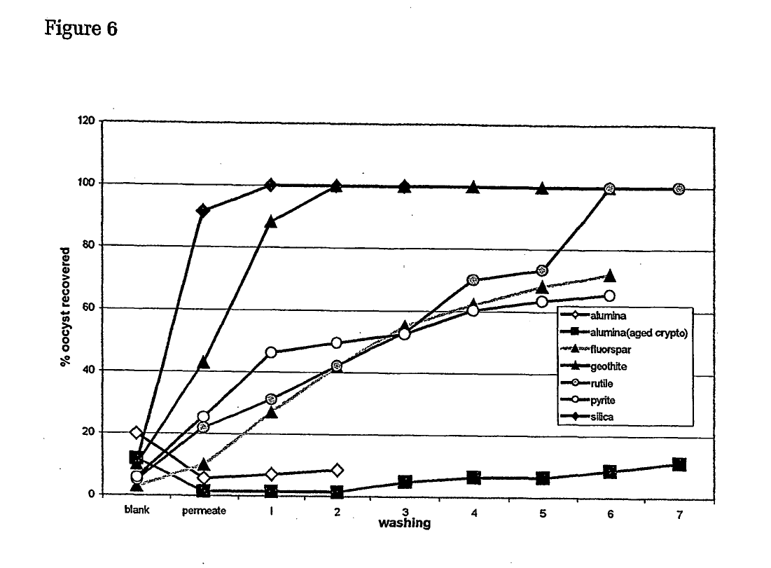

One pass of a dispersion of oocysts through the column of Al₂O₃ particles at a flow rate of 10 ml/1.28 min resulted in their complete removal from water. A further 10 distilled water washes of the Cryptosporidium loaded column with distilled water resulted in virtually no release of particulate material from the Al₂O₃ column (see FIG. 6). This indicated that the oocysts were strongly attached to Al₂O₃ particles.

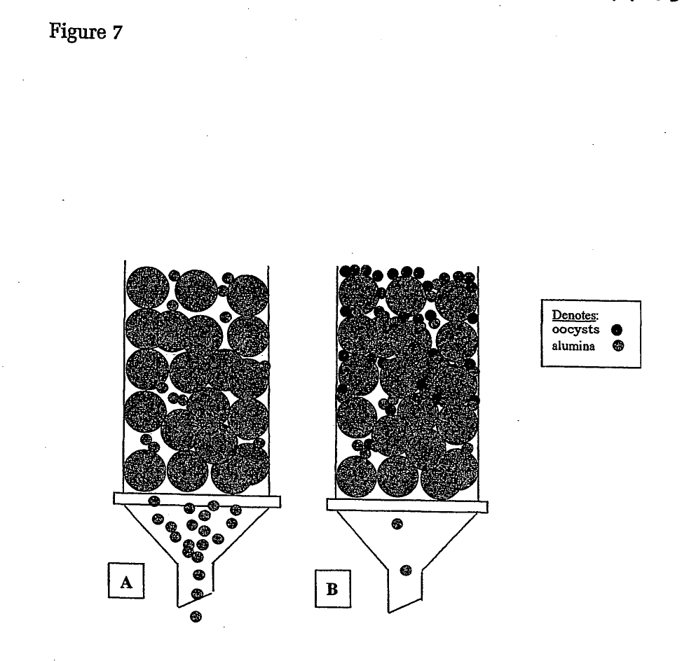

It is interesting to note that the number of particles for subsequent washings were well below that of background levels. Interestingly, this suggested that oocysts actually reduce the release of particulate material from the column. It is possible that finer particles which are normally released from the column are adsorbed onto the oocysts which are then adsorbed to the alumina support (see FIG. 7).

At the pH (5.5-6.0) at which column experiments were performed the Al₂O₃ surface would be positively charged. The Cryptosporidium oocysts would have a negative charge. Therefore, initially the oocysts would be adsorbed to the Al₂O₃ surface via an electrostatic interaction followed by specific chemisorption of the groups present on the oocyst with the hydrated AlOH groups on the Al₂O₃ surface.

At pH 7.0 the zeta potentials of Al₂O₃ and the oocysts were −17 and −27 mV, respectively. Column experiments carried out at this pH, however, also resulted in complete removal of the oocysts from water. Passage of water through the loaded column resulted in no release of the oocysts. This result appears to suggest a specific irreversible interaction between the sites on the oocyst surface and on the Al₂O₃ surface.

Marklund et al., 1989 (Acta Chem. Scand. 43: 641-646) reported that the bond energy strength between carboxylic groups and Al species in aqueous solution was around 10 kT per bond. If it is assumed that the area of contact of the oocysts with a flat surface of Al₂O₃ is around 0.1 μm² it could be estimated that around 10³ chemical bonds could be formed. Therefore, the total energy of all the bonds that could potentially be formed at the interface between the oocysts and on the surface of Al₂O₃ could be in the range of 10³ kT.

The alumina column experiments were also repeated using an aged oocyst sample. This was important to ascertain whether oocyst capture was adversely effected by the age of the oocysts. Aluminium oxide 90 neutral 70-230 mesh (Merck) 63-200 microns was fractionated in a measuring cylinder to give the >100 micron fraction. A column was packed to a depth of 1 cm. The aged Cryptosporidium oocysts was loaded onto the packed column, the permeate collected and then the column flushed with copious distilled water washings. The permeate and each distilled water fraction collected and examined in a Zeta meter (Rank Bros microelectrophoresis MK11). The oocysts were found to be completely removed by the adsorbent, this result confirmed oocyst capture/removal was unaffected by the age of the oocyst.

To illustrate the specific effect of the alumina, column investigations were carried out for a number of other natural minerals: geothite (alpha-FeO.OH) and haematite (Fe₂O₃), fluorspar (CaF₂), pyrite (FeS₂), pyrolusite (MnO₂), rutile (TiO₂), silica (SiO₂), and quartz (see FIG. 6).

Goethite (Alpha-FeO.OH) Column

The charging behaviour of geothite powder used in the column experiments was determined as a function of pH by microelectrophoresis. The geothite surface was positively charged below pH 3.5 (i.e. the iso-electric point), at higher pH values, the alpha-FeO.OH surface became increasingly more negatively charged. The negative potential increased between pH values 3.8-7 (pH 6:−14 mV) in a linear fashion, then remained at a maximum value of −19 mV above pH 8.5. The iso-electric point measured (˜pH 3.5) was in agreement with that reported for a natural mineral geothite by Schuylenbrogh and Sanger (zpc pH 3.2).

The passage of the oocyst dispersion through the goethite column at pH between 5.5 and 6.0 resulted in no removal from water, i.e. the oocysts were not adsorbed on the mineral surface (see FIG. 6). An electrostatic repulsion would exist between geothite and oocyst surfaces opposing adsorption. However, alumina was found to completely remove oocysts even against this type of electrostatic repulsive barrier (i.e at pH 7 the Al₂O₃ surface possessed a potential of −17 mV and the oocysts −27 mV).

The reason for the lack of removal of oocysts by goethite compared with Al₂O₃ is unclear at this stage. Both Fe(III) and Al(III) would react with the active groups on the oocysts. Under the charge conditions determined by pH 6.5, however, would suggest that only the AlOH groups on Al₂O₃ would react with carboxylic (and phosphate) groups.

Haematite (Fe₂O₃) Column

The charging behaviour of haematite (Fe₂O₃) used in the column experiments were determined as a function of pH by microelectrophoresis. The haematite surface charge was positively charged below pH 3.0 (i.e. isoelectric point). At higher pH values the (Fe₂O₃) surface potential became increasingly more negatively charged. The isoelectric point measured (˜pH 3) was not in agreement with that previously reported by (iep˜pH 8.5±0.2).

Unfortunately column experiments could not be conducted due to continuous desliming of the haematite. However, given that the haematite surface is similar to that of geothite no oocyst removal was expected.

Fluorspar (CaF₂) Column

Fluorspar (fluorite) CaF₂ is a positively charged material at neutral pH conditions, for this reason fluorspar, like alumina should electrostatically bind oocysts. Chemisorption could also occur via the formation of a calcium carboxylate/phosphate with the oocyst surface.

The fluorspar obtained in rock form from the ANU Department of Geology, was coarsely ground in a ball mill and fractionated by sedimentation to remove any fine particles. The coarse fractionated material was transferred to a chromatography column (28 mm OD, No. 3 glass sinter) to a depth of 2 cm. The background particle density (blank) was determined by passing distilled water through the fluorspar support and collecting the permeate for examination in the microelectrophoresis cell.

100 μl of a 1×10⁷ oocysts/ml gamma irradiated Cryptosporidium parvum sample was diluted to 20 ml in distilled water. 10 mils of this solution was allowed to percolate through a 2 cm fluorspar packed column at a rate (10 ml/1.28 min), the permeate and several washings were collected and examined by dark field illumination in the microelectrophoresis cell, noting the particle density at each plane across the cell.

Even though the original Cryptosporidium feed had a high negative potential, and the column support should have a positive potential, oocysts still were found to come through in the initial permeate. The permeate was found to have dropped from an average particle density of 30 to 3, indicating that electrostatic forces acted to retain the oocysts to some degree within the column support The column was rinsed with a number of 10 ml distilled water washings, which appeared to show continuous particle desorption with each washing (see FIG. 6).

These initial results demonstrated oocysts were desorbing from the support However, the fluorspar sample used appeared to be quite violet in colour and it is possible the sample was impure. To ascertain the purity of the sample XRF (see Table 1) and microelectrophoresis was carried out.

The charging behaviour of fluorspar (CaF₂) used in the column experiments were determined as a function of pH by microelectrophoresis. The fluorspar surface charge was positively charged below pH 5.6 (i.e isoelectric point). At higher pH values the (CaF₂) surface potential became increasingly more negatively charged. The isoelectric point measured was found to be ˜pH 5.6.

TABLE 1
Chemical composition of the fluorspar sample used in the column
experiments. Determined by semi quantitative XRF.

| Oxide wt % | Wt % dry basis | Oxide wt % | Wt % dry basis |
|---|---|---|---|
| SiO₂ | 2.33 | MnO₂ | 0.01 |
| TiO₂ | 0.01 | MgO | 0.05 |
| Al₂O₃ | 0.1 | CaO | 60.5 |
| Fe₂O₃ | 0.02 | Na₂O | 0.01 |
| SO₃ | 0.5 | K₂O | 0.02 |
| Fluoride | 33-35 | P₂O₅ | 0.005 |
| PbO | 2.1 | Loss of ignition | 2.35 |

Pyrite (FeS₂) Column

Pyrite was shown to be ineffective as an adsorbent for Cryptosporidium removal, as oocyst desorption was found to occur continuously (see FIG. 6).

Pyrolusite (MnO₂) Column

Unfortunately column experiments could not be conducted, as the natural sample obtained consisted of needles ≦75 μm in size, which continuously deslimed upon washing.

Rutile (TiO₂) Column

The rutile used in these investigations (I.D. 46065) was obtained from the Department of Geology (The Faculties) at the ANU. It was sieved through a series of (75, 90, 105, 106 and 210 μm mesh) sieves.

The coarse 106-210 μm fraction was washed with copious distilled water to remove any fine particles then transferred to a chromatography column (28 mm OD, No. 3 glass sinter) to a depth of 2 cm. The background particle density (blank approx 3/field of view) was determined by passing distilled water through the rutile support and collecting the permeate for examination in the microelectrophoresis cell.

100 μl of a 3×10⁸ oocysts/ml gamma-irradiated Cryptosporidium parvum sample was diluted to 25 ml in distilled water. 5 mls of this solution was then transferred to the 2 cm packed rutile column, the solution allowed to stand 15 mins before being allowed to percolate through at a rate (10 ml/1.28 min). After the entire contents had passed through, the column was washed three times with 10 ml of distilled water allowing it to percolate through at a similar rate to that of the original Cryptosporidium sample. The permeate and each 10 ml fraction were collected, and examined in the microelectrophoresis cell using dark field illumination noting the particle density at each plane across the cell.

The Cryptosporidium sample, before passing through the packed support, contained about 64 negatively charged particles at a given plane of view within the cell. The permeate which was passed through the packed column was seen to have about 14 negatively charged particles. Several 10 ml washings were passed through the column to see whether the Cryptosporidium oocysts would desorb or whether there was a lag time involved in total “Cryptosporidium” recovery. Washings 1-7 were found to contain 6, 7, 7, 11, 2, 32 and 13 negatively charged particles, respectively. The results obtained, suggests the oocysts were only weaked retarded within the column and upon sequential washing the oocysts were found to desorb and pass continously through the column support (see FIG. 6).

Ballotini (Glass) Particle and Quartz

To mimic the composition of sand filters, glass (amorphous silica) and quartz (crystalline silica) particles were used as adsorbents for Cryptosporidium oocysts. Both glass and quartz are negatively charged at pH values greater than 2 due to the ionisation of the surface silanol (—SiOH) groups.

The silica (Ballontini) spheres were fractionated to a size approx 200 mm in diameter. A slurry of these spheres were transferred to a glass chromatography column (28 mm O.D) containing a #3 glass sinter the excess liquid was run from the column leaving a packed column to a depth of 5 mm. 10 ml of distilled water was allowed to percolate through the packed column and the permeate collected. The permeate was transferred to the microelectrophoresis cell allowing the background level (8 negatively charged particles) to be determined. A 100 μl of a 3×10⁸ Cryptosporidium oocysts/ml was diluted to 25 mls in a volumetric flask, 10 ml of this solution was then transferred to the packed silica column, and the solution allowed to stand 15 mins before being allowed to percolate through the silica at a rate (10 ml/hr) into glass vials. After the entire contents had passed through, the column was washed three times with 10 ml of distilled water allowing it to percolate through at a similar rate to that of the original Cryptosporidium sample. Each 10 ml fraction was collected, transferred to a microelectrophoresis cell and examined using dark field illumination.

The Cryptosporidium sample before passing through the packed support contained about 75-78 negatively charged particles at a given plane of view within the cell. The permeate which was passed through the packed column was seen to have about 70 negatively charged particles. Three further 10 ml washings were put through the column to see whether the Cryptosporidium oocysts would desorb or whether there was a lag time involved in total “Cryptosporidium” recovery. The first, second and third washings were found to contain 34, 13 and 8 negatively charged particles, respectively. The results obtained are shown graphically in FIG. 6. This demonstrates that Cryptosporidium was not retained in the column support (i.e no adsorption evident) thus allowing the oocysts to travel through large intra-pore spacing between adjacent silica particles. The Cryptosporidium oocysts were found to pass easily through both quartz and Ballontini columns.

The charging behaviour of silica (SiO₂) used in the column experiments were determined as a function of pH by microelectrophoresis. The silica surface charge was positively charged below pH 2 (i.e. the isoelectric point). At higher pH values the (SiO₂) surface potential became increasingly more negatively charged.

### Example 6

Fluorescent Staining and Epifluorescence Investigations

Fluorescent staining and epifluorescence microscopy techniques were carried out to study the effects of Cryptosporidium oocysts adsorption to plasma cleaned silica (i.e. blank) and sapphire wafers. Sapphire (α-alumina) having a similar surface to that of CRyptoBlast™ (hydrated aluminium oxide/alumina) was used to develop a fundamental understanding of the surface governing the interaction between oocysts and the CRyptoBlast™ surfaces. It appears that the mode of action of this anti-cryptosporidial compound is by promoting adsorption to sapphire or CRyptoBlast™ substrates which led to oocyst destruction.

Distilled water seeded with C. parvum oocysts was loaded onto a laboratory scale (1.5 cm) column, packed with (150-200 micron) CRyptoBlast™ to a depth of 2.3 mm. The water was allowed to elute through the column, followed by several distilled water washes, the eluant fractions were collected and analysed. The CRyptoBlast™ column was found to remove 99.996% (>4-logs) of C. parvum oocysts in the process. The results obtained at the laboratory scale show high efficiency given the high oocyst load, the small packing depth and the high flow rate.

By contrast, comparable columns loaded with glass (silica) (Ballotini) spheres with a particle size of around 80 microns only removed between 31-51% of the oocysts (ie 0.2-0.3 log removal). The removal of oocysts with the glass spheres correlates with literature reports that indicate that oocysts can be partially removed with a sand filter. However, it must be stressed that any removal by silica could only occur via filtration as silica is unable to chemisorb oocysts, due to the lack of specific inteaction between the silanol groups and the oocysts surface. In addition both oocysts and silica are negatively charged at pH above around 2.5. Therefore, removal from water could not be attributed to electrostatic attraction.

Epifluorescence microscopy of oocysts stained with CRY104, a proprietary, Cryptosporidium-specific, fluorescent, monoclonal antibody, showed oocyst destruction was unaffected by the surface modification resulting from the staining procedure.

In order to gain further insight into the impact of CRyptoBlast™ on oocyst viability, fluorescent in-situ hydridization (FISH) testing and staining with the fluorogenic vital dye, 4′,6-diamidino-2-phenylindole (DAPI) was carried out. Both tests involve procedures that permeabilize the oocyst wall.

The FISH probes penetrate the oocyst wall and bind to 1000s of specific rRNA targets in the 4 sporozoites contained within the oocyst. DAPI also penetrates the oocyst and targets the nucleii and DNA.

Oocysts which were treated with CRY104, FISH and DAPI and then applied to the sapphire (α-alumina) wafer initially gave positive CRY104, positive FISH and positive DAPI signals. However, after half hour although the CRY104 signal was still positive, the FISH and DAPI probes became negative indicating the RNA and DNA of the sporozoites were destroyed in the adsorption process.

It appears likely that the oocyst permeabilization used for FISH/DAPI prevented the oocyst destruction from being as explosive as usually seen on non-permeabilized oocysts. As the oocyst shell remained partially intact (i.e a ghost cell) a positive CRY104 signal was observed. However, with time (>0.5 hr) the oocyst contents, i.e the sporozoites were released, and their RNA/DNA destroyed (FISH negative, DAPI negative) by the adsorption process onto “CRyptoBlast”.

These and the earlier column experiments indicate that columns of fine “CRyptoBlast™” particles can be used for the effective removal of oocysts from swimming pool water (as a polishing filter) and drinking water (as a household tap filter). The alternative methods, such as diatomaceous earth filters, have much smaller particle sizes, since they operate via physical filtration, and therefore require much higher pumping pressures and would give much lower flow-rates. The active adsorption filtration process, inherent in the “CRyptoBlast™” method, offers removal under much more practical conditions with greater commercial value.

### Example 7

Animal Infectivity Experiments

To test the effect of CRyptoBlast™ on the infectivity of live oocysts, animal infectivity experiments were carried out on 4 day old C57BL/6J neonatal and on adult interferon-gamma (IFN-γ) knockout mice to establish whether:

1. water infected with Cryptosporidium parvum when passed through a column of CRyptoBlast™ was non-infectious to oral ingestion.

2. treatment with CRyptoBlast™ prevents or decreases severity of infection in infected mice.

### Water Infected with C. Parvum Passed Through CryptoBlast™ Column

A 2 ml suspension of oocysts (˜1×10⁶/ml) was passed through a column (glass Pasteur pipette) containing a 3 cm high layer of fractionated CRyptoBlast™ (approx 150-200 micron particle size). Several samples from this filtrate were examined using a haemocytometer; no oocysts were detected in these samples. The filtrate from this treatment was then administered to the neonatal 4 day old C57BL/6J mice to see if an infection would establish. Seven days post inoculation, all animals were sacrificed and the small intestines removed. Two samples from each experimental group were fixed for histology, with the remaining samples homogenised. The homogenates were examined for the presence of oocysts using a haemocytometer.

We found that the infected controls had typically ˜10⁵ oocysts in the intestinal homogenates. We could not detect any oocysts upon microscopic examination of intestinal samples in 50% of intestinal homogenates examined (total n=42) from mice inoculated with the CRyptoBlast™ treated filtrate, suggesting no infection had established. In the other 50% of samples, it was unclear whether low numbers of particles detected were oocysts or not These samples were concentrated further for examination, a great deal of debris in the samples combined with the possibility that there were only very low levels of oocysts present made it difficult to confirm whether or not infection was present. Samples were later examined by flow cytometry and found to contain 6, 5,108, 0 and 20 oocysts whereas, the infective controls were found to have in the order of 10⁵ oocysts.

The CRyptoBlast™ filter system removed oocysts effectively and appeared to prevent infection in mice inoculated with the CRyptoBlast™ treated filtrate. The lack of infection in the inoculated mice was confirmed by histological examination on intestinal samples. Direct examination of intestinal homogenates indicated either no oocysts present or very low numbers when detected. This indicates that use of the fine CRyptoBlast™ columns for swimming pool and household water treatment will not introduce infective by-products into the eluent.

### Therapeutic Use of CryptoBlast Using Neonatal Mice

4-5 day old C57BL/6J mice were infected with ˜10⁴-10⁵ oocysts. At day four of infection, when oocysts are being produced in the intestines, several neonatal groups were treated twice daily with a suspension of CRyptoBlast™ (18-32 micron particle size). These animals were treated for five days in this manner, by which stage (7-9 days post infection), an infection should have established. The mice were then sacrificed at the ninth day of infection and the small intestines removed for further analysis. From histology and direct heamatocytomer examination it appears CRyptoBlast™ reduced the level of infection.

### Therapeutic Use of CryptoBlast™ Using Adult Interferon-gamma (IFN-γ) Knockout Mice

The aim of these experiments was to determine if treatment with CRyptoBlast™ prevents, or decreases the severity of infection, in infected, immunodeficient mice. IFN-γ knockout mice are reported to be highly susceptible to infection, in that they can become infected with as little as 10 oocysts, due to their suppressed immune system. This susceptibility is reported to result in high levels of infection, where the entire length of the small intestines and adjacent organs become heavily colonised by parasitic stages. This colonisation results in rapid weight loss and high levels of oocyst shedding.

6-8 week old male C57BL/6J IFN-γ knockout mice were infected with ˜10⁴ oocysts. At day four of infection it was predicted, based on published data, that the infected mice would begin shedding oocysts in their faeces. It is at this stage (4 days pi), that treatment with 200 μl CRyptoBlast™ (18-32 micron particle size) will begin twice daily. This treatment was continued for 5 days, at which point (9 days pi), the animals were sacrificed as the infection should be well established. Over the course of the experiment, infected controls showed no clinical signs of infection. No weight loss or oocysts in the faeces were detected. The experiment was left to continue for several more days until 20 days post infection at which point the experiment was terminated and the animals sacrificed. The entire small intestine from each animal, treated animals and controls, were removed for further analysis as done for the neonates. On examination of partial intestinal homogenates, the infective controls were found to be infected and the treated mice appeared to have a reduced level of infection. CryptoBlast™ treatment has shown to decrease the level of infection, in infected, immunodeficient and neonatal mice.

### Discussion and Conclusions

Cryptosporidium oocysts are irreversibly attached to alumina surfaces at pH values between 5 and 8. The adsorption is probably initially mediated by electrostatic attraction between negatively charged surface groups (most likely carboxylate and phosphate) on the oocysts followed by the stronger chemisorption between these groups and the specific sites on the surface of alumina. The strength of the adsorption is sufficient to rupture the oocysts exposing the sporozoites. Oocyst debris, including sporozoites, appear to be strongly captured by the alumina surface during the process.

By comparison, several other common natural materials such as goethite, haematite, quartz, silica, pyrite and fluorspar were all found to be completely ineffective as adsorbents for the oocysts.

The animal infectivity studies have indicated that compositions of the invention can be used as a therapeutic for the treatment of protozoal diseases, such as cryptosporidioisis. The hydrated aluminium oxide/alumina (e.g CRyptoBlast™) seems to work by “direct active adsorption”. Cryptosporidium is a coccidian enteric protozoan parasite infecting the animal by anal/oral route. Unlike other parasites Cryptosporidium usually continues its life cycle within the gastrointestinal tract rather than via infection in the blood stream. Oocysts ingested attach to the surface of the intestinal wall (in the upper intestine), where they undergo a complex life cycle. The oocysts multiply several fold in the infected host and are excreted in the faeces. Illness arises from the population growth on the walls of the upper intestine causing diarrhoeal disease; oocysts completing their life cycle are excreted in the faeces to potentially infect other hosts.

As CRyptoBlast™ has been shown to work via direct contact, CRyptoBlast™ given orally would progress through the gastrointestinal tract allowing it to come into direct active contact with Cryptosporidium ooocysts attached to the intestinal wall. Consequently, an ‘active-contact’ treatment method (as proposed) could reasonably be expected to have a beneficial effect on infected animals either via removal, or reduction of oocyst numbers to a level at which the body's natural immune defences can control the infection. The results presented indicate that the level of infection has been reduced by the use of CRyptoBlast™ as a therapeutic treatment for cryptosporiosis.

The alumina adsorbent has other potential uses (refer FIG. 1) such as a powdered adsorbent incorporated into baby nappy liners and baby waders, in drinking straws, drinking water cartridges (domestic units, water utilities), as a cartridge for water treatment backwash and recreational swimming pools. The adsorbent could also be used as a method of producing a high standard of water quality for use in pharmaceuticals preparations, food production including aquaculture. Alumina could be used in therapeutic agents for the treatment of protozoal diseases such as cryptosporidiosis or used in pharmaceutical preparations, as an alternative disinfectant for protozoans such as Cryptosporidium and Giardia and as a final stage filtration cartridge for the treatment of sewage effluent. In addition it could be used as prophylaxis in intensive farming (e.g. cattle, poultry etc)—such as neonatal calves, who are routinely infected with cryptosporidiosis. A slurry or dispersion of fine (micron sized) alumina particles in water, given orally would scavenge the oocysts and therefore allow agricultural animals to bulk up at a much earlier age.

Uses in agribusiness may include treatment of farm animal effluent and wastewater to minimise risk of contamination of water tables and rivers, as well as in the treatment of water used for cleansing vegetables and fruits, that are consumed without any further cooking/processing.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features, as appropriate.

1. A method of destroying oocysts, and destroying or inactivating sporozoites contained within the oocysts, which method comprises:
(iii) contacting the oocysts with a composition comprising surface-exposed AlOH groups which adsorb the oocysts causing the adsorbed oocysts (a) to rupture and the contained sporozoites to be released and/or
(b) to flatten resulting in destruction of the contained sporozoites; and, optionally
(iv) contacting the released sporozoites with an effective amount of an anti-sporozoidal agent.

2. A method of destroying an oocyst, and destroying or inactivating one or more sporozoites contained within the oocyst, which method comprises contacting the oocyst with a composition comprising surface-exposed AlOH groups to cause the oocyst to rupture and the contained sporozoites to be released such that the released sporozoites are brought into contact with the surface-exposed AlOH groups.

3. A method according to claim 2 which further comprises contacting the released sporozoites with an effective amount of an anti-sporozoidal agent.

4. A method according to claim 1 or 2 wherein the surface-exposed AlOH groups are present as hydrated alumina.

5. A method according to claim 1 or 2 wherein the exposed AlOH groups are present at an average surface density of greater than about 1 hydroxyl group per 10 nm² of surface area.

6. A method according to claim 5 wherein the exposed AlOH groups are present at an average surface density of greater than about 1 hydroxyl group per 2 nm².

7. A method according to claim 6 wherein the exposed AlOH groups are present at an average surface density of greater than from about 1 hydroxyl group per 0.25 nm² to about 1 hydroxyl group per 0.18 nm².

8. A method according to claim 1 or 2 wherein the composition is in particulate or plate form.

9. A method according to claim 8 wherein the composition comprises particles which comprise surface-exposed AlOH groups and said particles have a diameter in the range of from about 15 mm to about 0.05 mm.

10. A method according to claim 9 wherein the particles which comprise surface-exposed AlOH groups have a diameter in the range of from 1.5 mm to about 0.05 mm.

11. A method according to claim 1 or 2 wherein one or more of the oocysts are Cryptosporidium or Giardia cysts.

12. A method according to claim 11 wherein one or more of the oocysts are Cryptosporidium oocysts.

13. A method according to claim 1 or 2 wherein the anti-sporozoidal agent is selected from chlorine and ozone.

14. A method according to claim 1 or 2 wherein the oocysts are present in a fluid.

15. A method according to claim 14 which further comprises purifying the fluid to remove the sporozoidal agent and/or composition.

16. A method according to claim 15 wherein the fluid is a biological fluid.

17. A method according to claim 15 wherein the fluid is water.

18. A method according to claim 17 wherein the water is intended for use by humans or animals.

19. A method according to claim 17 wherein the water is intended for human or animal consumption.

20. A method according to claim 17 wherein the water is intended for the preparation of food products or pharmaceutical products.

21. A method according to 20 wherein said food products are, or are derived from, aquatic organisms, and the aquatic organisms are grown in the water.

22. A pharmaceutical composition comprising a composition as defined in claim 1 or 2 together with a pharmaceutically acceptable carrier or diluent.

23. A food supplement comprising a composition as defined in claim 1 or 2.

24. A method of treating, preventing or reducing a protozoal infection in a human or animal, said protozoal infection being initiated by the ingestion of oocysts, which method comprises administering to said animal or human a pharmaceutical composition according to claim 22.

25. A method according to claim 24 which further comprises administering an anti-sporozoidal agent to the animal or human.

26. A food receptacle, drinking receptacle or drinking straw comprising a composition as defined in claims 1 or 2 such that when in use, the composition contacts food or fluid placed in the receptacle or drawn through the drinking straw.

27. An item of clothing selected from a nappy, underwear or swimwear comprising a composition as defined in claim 1 or 2 such that when the clothing is worn by an individual, the composition is brought into contact with faecal matter produced by the individual.

28. An item of clothing according to claim 27 wherein the clothing further comprises an anti-sporozoidal agent.

29. A method for producing a pharmaceutical composition which method comprises admixing a composition as defined in claim 1 or 2 with a pharmaceutically acceptable carrier or diluent.

30. A method of destroying or inactivating a pathogenic proteinaceous agent which method comprises contacting the agent with a composition comprising surface-exposed AlOH groups to cause the agent to be adsorbed to the surface-exposed AlOH groups.

31. A method of treating, preventing or reducing a protozoal infection in a human or animal, said protozoal infection being initiated by the ingestion of oocysts, which method comprises administering to said animal or human a food supplement according to claim 22.
